(19) United States
(12) Patent Application Publication
(10) Pub. No.: US 2003/0003489 A1
(43) Pub. Date: Jan. 2, 2003

(54) Combinatorial peptide expression libraries using suppressor genes

(76) Inventors: Paul Theodore Hamilton, Cary, NC (US); Robin Parish Hyde-DeRuyscher, Chapel Hill, NC (US)

Correspondence Address: BROWDY AND NEIMARK, P.L.L.C., 624 Ninth Street, N.W., Washington, DC, 20001, US

(21) Appl. No.: 10/154,932

(22) Filed: May 28, 2002

Related U.S. Application Data
(63) Continuation of application No. 09/391,601, filed on Sep. 8, 1999, now abandoned.
(60) Provisional application No. 60/099,655, filed on Sep. 9, 1998.

(51) Int. Cl. C12Q 1/68; G01N 33/53; C12P 21/02; C12N 5/06

(52) U.S. Cl. 435/6; 435/7.1; 435/69.1; 435/325; 435/320.1

(57) ABSTRACT

The biased residue of an expressible biased peptide library is conveniently altered, without synthesizing a new DNA mixture, by using a DNA encoding said peptide which includes a suppressible stop codon, said codon encoding the biased residue, whereby the amino acid appearing at the biased position may be altered simply by introducing the same DNA mixture into a different suppressor strain.

### BACKGROUND OF THE INVENTION

1. Field of the Invention

This invention relates to combinatorial peptide libraries produced by expression of a randomized gene, and to the use of such libraries in screening peptides for the ability to specifically bind a target substance.

2. Description of the Background Art

Protein Binding and Biological Activity

Many of the biological activities of the proteins are attributable to their ability to bind specifically to one or more binding partners (ligands), which may themselves be proteins, or other biomolecules.

When the binding partner of a protein is known, it is relatively straightforward to study how the interaction of the binding protein and its binding partner affects biological activity. Moreover, one may screen compounds for the ability of the compound to competitively inhibit the formation of the complex, or to dissociate an already formed complex. Such inhibitors are likely to affect the biological activity of the protein, at least if they can be delivered in vivo to the site of the interaction.

If the binding protein is a receptor, and the binding partner an effector of the biological activity, then the inhibitor will antagonize the biological activity. If the binding partner is one which, through binding, blocks a biological activity, then an inhibitor of that interaction will, in effect, be an agonist.

The residues whose functional groups participate in the ligand-binding interactions together form the ligand binding site, or paratope, of the protein. Similarly, the functional groups of the ligand which participate in these interactions together form the epitope of the ligand.

In the case of a protein, the binding sites are typically relatively small surface patches. The binding characteristics of the protein may often be altered by local modifications at these sites, without denaturing the protein.

While it is possible for a chemical reaction to occur between a functional group on a protein and one on a ligand, resulting in a covalent bond, protein-ligand binding normally occurs as a result of the aggregate effects of several noncovalent interactions. Electrostatic interactions include salt bridges, hydrogen bonds, and van der Waals forces.

What is called the hydrophobic interaction is actually the absence of hydrogen bonding between nonpolar groups and water, rather than a favorable interaction between the nonpolar groups themselves. Ringe suggests that a large part of the binding energy for protein-ligand interactions is due to the displacement of water (Ringe. 1995. What makes a binding site a binding site. Current opinion in Structural Biology 5:825-829).

Combinatorial Libraries

Libraries of thousands, even millions, of random oligopeptides have been prepared by chemical synthesis (Houghten et al., Nature, 354:84-6(1991)), or gene expression (Marks et al., J Mol Biol, 222:581-97(1991)), displayed on chromatographic supports (Lam et al., Nature, 354:82-4(1991)), inside bacterial cells (Colas et al., Nature, 380:548-550(1996)), on bacterial pili (Lu, Bio/Technology, 13:366-372(1990)), or phage (Smith, Science, 228:1315-7(1985)), and screened for binding to a variety of targets including antibodies (Valadon et al., J Mol Biol, 261:11-22(1996)), cellular proteins (Schmitz et al., J Mol Biol, 260:664-677(1996)), viral proteins (Hong and Boulanger, Embo J, 14:4714-4727(1995)), bacterial proteins (Jacobsson and Frykberg, Biotechniques, 18:878-885(1995)), nucleic acids (Cheng et al., Gene, 171:1-8(1996)), and plastic (Siani et al., J Chem Inf Comput Sci, 34:588-593(1994)).

Libraries of proteins (Ladner, U.S. Pat. No. 4,664,989), peptoids (Simon et al., Proc Natl Acad Sci U S A, 89:9367-71(1992)), nucleic acids (Ellington and J W, Nature, 246:818(1990)), carbohydrates, and small organic molecules (Eichler et al., Med Res Rev, 15:481-96 (1995)) have also been prepared or suggested for drug screening purposes.

The chemistry of peptide libraries is quite similar to many of the natural macromolecules involved in biological processes and thus these libraries are rich in structures that mimic the natural ones which interact with the target protein. In addition, the variants are composed of linear polymers such that each actually represents a sliding window of many differing chemical constituents. For instance, if a given macromolecular interaction is based on the side chains of four amino acids within a binding peptide, then a 13 amino acid peptide has 10 potential combinations of residues which may bind; therefore a library of 10⁸ members has about 10⁹ 4-mer permutations. This, combined with ease of producing and screening exceptionally large and diverse peptide libraries, provides the incentive to use peptide combinatorial libraries for the initial identification and probing of protein functional domains.

Peptide libraries provide a diverse source of chemical shapes with many functions. The libraries can be made synthetically or are encoded by nucleic acids. Genetically encoded peptide libraries can be made in a number of systems, with phage display, polysome display, bacterial display, lac repressor, baculovirus, and yeast two hybrid being the most commonly used. The advantage of using genetically encoded libraries vs synthetic libraries is that they can be amplified, which allows for multiple rounds of selection as well as the propagation of the library for future use.

Biased Libraries

The first oligopeptide-on-phage libraries randomly mutated all amino acid positions of the oligopeptide sequence in question such a library can be said to be “unbiased”, in the sense that any amino acid can occur at any position, although some amino acids may be more strongly represented as a result of the degeneracy of the genetic code.

It was recognized at an early date that if one had information about the binding preferences of the target, it could be advantageous to hold certain amino acid positions of the library peptides constant. For example, the constant residues could be a known part of the binding motif. These biased libraries are also called “purpose-built” libraries and numerous examples exist in the literature. See Sparks et al., Proc. Natl. Acad. Sci. USA 93:1540-1544 (1996), Sparks, et al., J. Biol. Chem. 269:23853-23856 (1994), Linn et al., Biol. Chem. 378:531-537 (1997). Indeed, once an unbiased peptide library has been screened, it is likely that subsequent libraries will be biased in the light of the knowledge previously gained. See Blake U.S. Pat. No. 5,565,325.

Biased libraries have also been prepared, without prior knowledge of the specific binding site of interest, but taking into account general information concerning the frequency of occurrence of particular residues in binding sites. See Fowlkes, WO98/19162. Another reason for holding certain residues constant is to constrain the conformation which the peptide can assume. See Ladner, U.S. Pat. No. 5,223,409, Example XII. For example, a peptide may have constant cysteines which can form a disulfide bond.

Biased libraries have also been prepared for reasons of synthetic convenience. See Rutter U.S. Pat. No. 5,010,575.

Pinilla, U.S. Pat. No. 5,556,762 suggests that it can be advantageous to prepare a “set” (panel) of biased peptide libraries where the biased position is the same for all of the libraries, and this position, while the same amino acid for all peptides within a given library, differs from library to library within the set as a whole. While Pinilla uses the term “scanning”, she accords it a different meaning than we do.

Gene Expression

In gene expression, a DNA-directed RNA polymerase binds to the promoter operably linked to the gene, and then traverses the gene, transcribing the DNA into RNA. It does this by synthesizing an RNA complementary to the noncoding strand of the DNA of the gene. This RNA is processed (introns removed) to yield a messenger RNA, which then acts as a template for the construction of the encoded polypeptide. In this process, which is called translation, amino acid-charged transfer RNAs bind by virtue of their anticodon to the complementary mRNA, and their amino acid is released and coupled to the nascent polypeptide chain.

The transfer RNAs have a nucleotide sequence which can be written in a “cloverleaf” form illustrating the presence of both base-paired stems and unpaired loops. Like polypeptides, transfer RNAs are encoded by genes, however, while their genes are transcribed into RNA, this RNA is not translated into protein. Transfer RNAs are not restricted in composition to the normal four bases (A, G, C, U); other bases may be produced by modification of the originally synthesized bases.

In order to play a role in protein synthesis, a transfer RNA must be. “charged” with the amino acid corresponding to its DNA-binding anticodon. This charging is catalyzed by specific enzymes called aminoacyl-tRNA synthetases. The tRNAs recognized by a given synthetase are called its cognate tRNAs.

The genetic code is composed of 64 triplet codons. In bacteria, and in most organisms, 61 of the codons code for the 20 amino acids and three codons, UAG, UGA, and UAA, are termination codons. Within the sequence of a protein-encoding gene, a mutation that results in a change from an amino acid to chain-termination is termed a nonsense mutation. Nonsense suppressors are mutations that alter tRNAs, e.g., by altering the anticodon, so as to allow them to insert an amino acid at a location specified by one of the termination triplets. Using a series of nonsense suppressors with the corresponding nonsense codon, a set of amino acid substitutions can be made at the position of the nonsense mutation in a protein. Miller and his colleagues have constructed synthetic suppressor genes in Escherichia coli and used them for nonsense suppression to study protein structure and function in E. coli (Miller et al., 1989; Kleina, et al., 1990; Normally et al., 1990; Miller, 1991).

Functional suppressor tRNAs have been described in bacterial, yeast (Liebman et al), Caenorhabditis (Kondo et al), Dictyostelium (Dingermann et al), plant (Franklin et al), Drosophilia (Laski et al, 1989), Xenopus (Bienz et al.), and mammalian systems (Laski et al, 1984).

Use of Suppressor Codons in Phage Libraries

Suppressible codons have been used in phage display technology to allow the expression of both the fusion protein (foreign peptide or protein-to-phage coat protein) and the wild type foreign peptide or protein from a single DNA construct. The gene is engineered so that a suppressible amber stop codon appears between the DNA encoding the foreign peptide or protein, and the DNA encoding the coat protein. In an amber suppressor strain, the fusion protein is expressed. In a non-suppressor strain, only the sequence up to the stop codon is expressed. For use of this approach, see Huse (1992), Lowman (1991), and Felici (1991).

This invention is the first to use suppressor codons for the purpose of varying the binding sequence.

All references, including any patents or patent applications, cited in this specification are hereby incorporated by reference. No admission is made that any reference constitutes prior art. The discussion of the references states what their authors assert and applicants reserve the right to challenge the accuracy and pertinency of the cited documents.

### SUMMARY OF THE INVENTION

The present invention relates to biased peptide expression libraries in which at least one biased amino acid position is encoded by a suppressible codon, so that the amino acid appearing at that position is dependent on whether the bacterial host cells in which the peptide library is expressed also expresses a corresponding suppressor gene. Thus, a single library could be constructed, and the amino acid appearing at the suppressor gene-mediated biased position would be dependent on which suppressor gene was co-expressed.

In a preferred embodiment, the suppressible codon is a stop codon, and, more preferably, an amber codon (TAG).

The suppressor stains could be used individually, in small groups or as a complete mixture. Using libraries that are biased at different positions, it should be possible to determine a consensus binding sequence without sequencing individual clones. Conversely, using a library with the amber codon scanning through the random region in conjunction with the different suppressor strains, it should also be possible to determine a consensus binding sequence.

In one embodiment, the library is a bacteria phage library, and the suppressor gene is chromosomally or extra chromosomally (e.g., plasmid) encoded by the host bacteria.

In another embodiment, peptide gene is carried by a yeast compatible vector, and the vector is introduced into yeast, the yeast co-expressing a suppressor gene.

### BRIEF DESCRIPTION OF THE DRAWINGS

FIG. 1 Raw Frequency of Residue Occurrence in Phage-Displayed Peptides

FIG. 2 Corrected Frequency of Residue Occurrence in Phage-Displayed peptides

### DETAILED DESCRIPTION OF THE PREFERRED EMBODIMENTS

Methods and compositions are provided for the construction of biased peptide libraries (as hereafter defined) in which a biased amino acid is encoded by a suppressible stop codons.

The biased residue is a residue which is fixed for all members of a given library. In conventional biased peptide libraries, to generate each new biased library, a new degenerate oligonucleotide cassette, with the biased codon changed to encode a different amino acid and, must be synthesized and cloned into the vector. Thus, preparation of a panel of twenty different biased libraries, differing in terms of the choice of amino acid at the biased position, but not-in terms of the location of the biased residue within the peptide, would conventionally require synthesis of twenty differently biased oligonucleotide cassettes.

The use of suppressor strains, such as the amber (TAG) suppressing strains of E. coli, would allow the synthesis of a single degenerate oligonucleotide cassette to generate such a panel of different biased libraries. In one embodiment, the biased library cassette would contain the TAG codon in the position of the desired bias. The cassette would be cloned into the appropriate vector. The amino acid present at the biased position would depend on the strain of E. coli used to propagate the library. For example, an E. coli strain with a supD genotype would insert serine in place of the TAG codon. While a strain with a supe genotype would insert glutamine in place of the TAG codon. Presently available amber suppressor strains allow the biased amino acid to be one of at least 14 different amino acids.

Library

The term “library” generally refers to a collection of chemical or biological entities which can be screened simultaneously for a property of interest. (They may be screened sequentially, if desired, but simultaneous screening is more efficient.) Typically, they are related in origin, structure, and/or function.

The term “combinatorial library” refers to a library in which the individual members are either systematic or random combinations of a limited set of basic elements, the properties of each member being dependent on the choice and location of the elements incorporated into it. Typically, the members of the library are at least capable of being screened simultaneously. Randomization may be complete or partial; some positions may be randomized and others predetermined, and at random positions, the choices may be limited in a predetermined manner, or the relative frequency of appearance of the allowed choices may be adjusted as desired. The ability of one or more members of such a library to recognize a target molecule is termed “Combinatorial Recognition”.

A combinatorial peptide library is a combinatorial library whose members are peptides having three or more amino acids connected via peptide bonds. The peptides may be linear, branched, or cyclic, and may include nonpeptidyl moieties. The amino acids are not limited to the naturally occurring amino acids. The peptides need not, but may, be of the same length. The individual peptides are referred to as peptide ligands (PL).

An “expressible peptide library” is one in which all component peptides are obtainable by expressing a gene encoding the peptide. Hence, the amino acids are limited to the 20 genetically encoded amino acids.

A “displayable peptide library” is one in which all the component peptides are either directly expressible, or can be obtained by chemical or enzymatic modification of the originally expressed peptide in situ, i.e., on the surface of a cell or virus.

A biased combinatorial library is one in which, at one or more positions in the library member, only one of the possible basic elements is allowed for all members of the library, i.e., the biased positions are invariant. A biased combinatorial peptide library is one in which, at one or more (but not all) biased residue positions (counted from the N-terminal) of the peptides, all peptides of the library exhibit the same amino acid, i.e., these biased positions exhibit “constant” residues. Typically, 1, 2, or 3 positions are variable positions, and indeed in the peptide are held constant, and the remaining positions can be any amino acid.

The biased library may be constructed by cloning an oligonucleotide mixture, which encodes the biased peptides, into copies of the appropriate expression vector. Ideally, each molecule of the oligonucleotide mixture is inserted into a different vector molecule. The oligonucleotide cassette used to construct the biased library encodes both variable residues and constant residues. The variable residues may be encoded by an (NNK)ₙ coding scheme, where N may be A,C,G, or T and K is G or T and each NNK codon encodes an amino acid in the peptide. The NNK codon encodes all twenty genetically encoded amino acids. Other codons are described in Ladner, U.S. Pat. No. 5,223,409. From 2-20 different amino acids can be represented at each variable position of an expressible peptide library.

A “panel of combinatorial libraries” is a collection of different (although possibly overlapping) and separately screenable.

A “structural panel” is a panel as defined above where there is some structural relationship between the member libraries. For example, one could have a panel of 20 different biased peptide libraries where, in each library, the middle residue is held constant as a given amino acid, but, in each library the constant residue is different, so, collectively, all 20 possible genetically encoded amino acids are explored by the panel.

A “scanning residue library” refers to the preparation of panel of biased combinatorial peptide libraries such that the position of the constant residue shifts from one library to the next. For example, in library 1, residue 1 is held constant as a particular residue AA, in library, residue 2 is, and so forth through two or more (usually all) positions of the peptide.

One may have structured panels of libraries in which one may define subpanels, too. For example, in one subpanel, the middle residue AA₁ may be the same for all libraries, but the libraries also have a constant residue AA₂ which is scanned through all other residue positions.

A library screening program is a program in which one or more libraries (e.g., a structured panel of biased peptide libraries) are screened for activity. The libraries may be screened in parallel, in series, or both. In serial screening, the results of one screening may be used to guide the design of a subsequent library in the series.

The size of a library is the total number of molecules in it, whether they be the same or different. The diversity of a library as the number of different molecules in it. “Diversity” does not measure how different the structures of the library; the degree of difference between two structures is referred to here as “disparity” or “dispersion”. The “disparity” is quantifiable in some respects, e.g., size, hydrophilicity, polarity, thermostability, etc. The average sampling frequency of a library is the ratio of size to diversity. The sampling frequency should be over the detection limit of the assay in order to assure that all members are screened.

The combinatorial libraries usually will have a diversity of at least 10different structures. Preferably, the initial, surrogate-generating library is of high diversity, e.g., preferably at least about 10⁶, more preferably at least about 10⁹ different members. While a peptide library is preferred, a library composed of a different class of compounds (e.g., peptoids or nucleic acids) is acceptable if there would be a detectable preference for binding the activity-mediating binding sites of the target protein.

Suppressor Systems

A nonsense suppressor system is an organism or a cell free expression system which, when expressing a DNA comprising a nonsense (TAG, TAA or TGA) codon, will place amino acid into the nascent polypeptide chain at the amino acid position corresponding to that nonsense codon, rather than interpreting it as a “stop” (termination) codon and terminating chain synthesis. An amber suppressor system suppresses the amber codon (TAG); ochre (TAA) and opal (TGA) suppressor systems are analogously defined. (The corresponding mRNA codons substitute U for T.)

The organism may be a prokaryotic or a eukaryotic cell. Preferred eukaryotes are yeast cells such as S. cerevisiae. The preferred prokaryotes are bacteria, and especially E. coli and S. typhimurium.

It is not necessary that the system suppress the nonsense codon in every molecule of messenger RNA read. Preferably the efficiency of suppression is at least 5%, more preferably at least 10%, even more preferably at least 50%, still more preferably at least 90%, most preferably at least 95%.

The efficiency of nonsense suppression is affected by the sequence surrounding the nonsense codon in the mRNA, especially the two bases following the codon in the case of amber (UAG) codons, the efficiency of suppression depends on the next base as follows: A>G>U, C. Suppression is strongest when the trailing codon is AUX. An exception to the rule that C reduces efficiency exists when this trailing codon is CUX.

Given the nature of the genetic code, when an amber codon is followed by an Leu, Ser, or Arg codon it is feasible to change that trailing codon to a codon favorable to amber suppression:

| | |
|---|---|
| Leu | CUX |
| Arg | AGA, AGG |
| Ser | AGU, AGC |

In the context of a phage display library, this means that the trailing codon should be randomized such that, if it encodes Leu, Arg or Ser, it does so via a suppression-favoring triplet.

If the efficiency is less than 100%, the library will contain some level of truncated peptide, i.e., consisting only of the peptide encoded by the mRNA up to the nonsense codon.

If a phage display system were used, this truncated polypeptide would not be incorporated into the mature phage and so would not interfere with the system. However, any fusion system where the peptide was a carboxy-terminal fusion would contain some truncated peptide.

It is possible that this fragment will bind to the target molecule. However, one may readily ascertain whether particular phage are bound by virtue of full-length or truncated peptide by (1) sequencing the displayed peptide, (2) transforming the recovered binding phage to a non-suppressor expression system, (3) using a non-suppressor expression system as a control, or (4) synthesizing and testing the putative binding peptide, and, optionally, the potentially competitive truncated peptide.

Another consideration is the specificity of the insertion. A suppressor system could insert just a single amino acid in every case, in which event it is absolutely specific. Or it could insert one of a small number of different amino acids. For example, one suppressor known in the art inserts Glu 80% of the time, and Gln 20%.

Preferably, at least for suppressors other than of Glu or Gln, the specificity of insertion is at least 95%, more preferably at least 99%. A lack of specificity causes the problem that the peptide deduced by sequencing the relevant DNA of a target-binding phage may not in fact be the target-binding peptide. However, since the invention contemplates placing the same phage library in a plurality of different suppressor systems, it should become readily apparent which peptide binds target most strongly.

The ability to suppress a nonsense codon is imparted by a suppressor tRNA gene. The following amber suppressor tRNA genes are available.

| Codon | | Amino acid | |
|---|---|---|---|
| Suppressed | Gene | inserted | reference |
| UAG | supD | Serine | Steege, (1983) |
| UAG | supE | Glutamine | Inokuchi et al., |
| | | | (1979) |
| UAG | supF | Tyrosine | Goodman et al., (1968) |
| UAG/UAA | supG | Lysine | Gorini, (1970) |
| UAG | supP | Leucine | Thorbjarnardottir et |
| | | | al., (1985) |
| | | | Yashimura et al., |
| | | | (1984) |
| UAG | glyT | Glycine | Prather et al., (1981) |
| UAG | Synthetic | Alanine | Normanly et al., |
| | tRNAala | | (1990) |
| UAG | Synthetic | Cysteine | Normanly et al., |
| | tRNAcys | | (1990) |
| UAG | Synthetic | Glutamic | Normanly et al., |
| | tRNAGluA | acid/Gluta | (1990) |
| | | mine | |
| UAG | Synthetic | Glycine | Normanly et al., |
| | tRNAGly1 | | (1990) |
| UAG | Synthetic | Histidine | Normanly et al., |
| | tRNAHisA | | (1990) |
| UAG | Synthetic | Lysine | Normanly et al., |
| | tRNALys | | (1990) |
| UAG | Synthetic | Phenylalanine | Normanly et al., |
| | tRNAPhe | | (1990) |
| UAG | Synthetic | Proline | Normanly et al., |
| | tRNAProH | | (1990) |
| UAG | Synthetic | Arginine | Normanly et al., |
| | FTOIRΔ26 | | (1990) |
| UGA | trpT | Tryptophan | Raftery et al., (1984) |

In E. coli, the amber suppressing genes represent the majority of the described suppressor genes. There are, however, also suppressor genes for the other termination codons in E. coli TAA-ochre and TGA-opal. Many of the ochre suppressors, unfortunately, suppress both TAA and TAG.

There has been some work to increase the number of available opal suppressors (McClain et al., 1990). This set is still less than the available amber suppressor strains. The availability of independent amber and opal suppressor genes would allow the construction of a strain expressing two suppressor genes, one amber and one opal. This makes it possible to construct a biased library that has two different suppressible codons at two different biased positions. Thereby producing a double biased library. In the extreme case of all 20 possible opal and amber suppressors, then a single oligonucleotide cassette with a single opal codon and a single amber codon could be used to generate 400 different double biased libraries.

Ochre suppressors unfortunately suppress both UAA and UAG. However, if exceptions are identified which suppress only ochre, they could be used.

For techniques of constructing nonsense suppressor mutants of normal transfer RNA genes, see Kleina, et al., J. Mol. Biol., 213:705-17 (1990); Normanly, et al., J. Mol. Biol., 213:719-26 (1990); Miller, et al., Genome, 31:905-8 (1989). New suppressor strains could be generated by mutagenesis and/or recombinant DNA techniques, as described by Miller (1991) and Martin et al., (1996).

While most of the suppressor systems which have been studied have been nonsense suppressors, it is also possible for a sense codon to be suppressed, so that whether that codon encodes are amino acid or another is dependent on whether expression occurs in a suppressor or a non suppressor strain. This type of suppression, which is called missense suppression, can occur as a result of altering either the anticodon, or the acceptor stem (and hence the charged AA), of the wild type transfer RNA. Suppressor mutants are known which cause the Gly codons GGG and GGA to be interpreted as arginine codons, or a glutamine residue to be transferred in response to the tyrosine codon UAG. Strong missense suppressors are rare because efficient substitution would have damaging effects on the function of other given.

Biasing of Residues

In a preferred biased peptide library embodiment, an internal residue is constant, so that the peptide sequence may be written as

(Xₐₐ)ₘ-AA₁-(Xₐₐ)ₙ

Where Xaa is either any naturally occurring amino acid, or any amino acid except cysteine, m and n are chosen independently from the range of 2 to 20, the Xaa may be the same or different, and AA₁ is the same naturally occurring amino acid for all peptides in the library but may be any amino acid. Thus, the peptides of this embodiment are 5-41 amino acids long. More preferably, m and n are chosen independently from the range of 4 to 9. Thus, the length of t-he more preferred peptides is 9 to 19 amino acids.

Preferably, AA₁ is located at or near the center of the peptide. More preferably, AA₁ is either (a) at least five residues from both ends of the peptide, or (b) is in the middle 50% of the peptide. More preferably, that m and n are not different by more than 2; most preferably m and n are equal. Even if the chosen AA₁ is required (or at least permissive) of the TP binding activity, one may need particular flanking residues to assure that it is properly positioned. If AA₁ is more or less centrally located, the library presents numerous alternative choices for the flanking residues. If AA₁ is at an end, this flexibility is diminished.

The most preferred libraries are those in which AA₁ is tryptophan (W), lysine (L), tyrosine (Y), phenylalanine, aspartic acid (D), and cysteine (C).

The effect of fixing one position in a library is to increase the occurrence of that particular residue from 1 in 20 to 20 in 20, an increase of 20 fold. Thus in theory if a particular residue is required for binding in the middle of the peptide, the rate of finding clones would be 20 fold higher than if a random residue were used. Therefore by using 20libraries with one fixed residue the chances of finding members that bind to the target protein would be increased [20×(# of residues conserved for binding)] when compared to using completely random libraries. These 20 libraries (or at least a subset of them) would be effective against any target and no prior knowledge of the sequence for the peptide ligand would be required.

Ligands that bind to functional domains tend to have both constant as well as unique features. Therefore, by using “biased” peptide libraries, one can ease the burden of finding ligands.

For example, HPQ occurs in most streptavidin-binding peptides, which bind with the HPQ side chains oriented inward so as to interact with the biotin-binding site of the TP streptavidin. Some of the residues that participate in binding biotin also interact with the peptides; however, the peptides adopt an alternate method of utilizing binding determinants (Biochemistry 31: 9350-4 (1992)[93003082], Crystal structure and ligand-binding studies of a screened peptide complexed with streptavidin, P. C. Weber, M. W. Pantoliano & L. D. Thompson). Therefore, if one starts off with a biased library e.g. X(6) —H—X (6), then one finds many binding peptides in a short period of time because that library will be rich in peptides having the cognate binding site.

The example above showed a biased library with one residue held constant. The net effect of this is to increase the number of peptides with the constant residue in that position. If this residue at this position is helpful for binding, then the number of individuals per library that will bind to the target protein will be increased. If all the amino acids are represented equally, then the number of potential binding peptides is increased 20 fold in a library made up of the 20 naturally occurring amino acids. Libraries using different ratios of amino acids will be enriched according to the proportion of each residue in the starting library.

Of course, if the library is biased with a constant residue which happens to disrupt binding, the screening results will be negative. Therefore, it may be advantageous to screen a plurality (a panel) of different biased peptide libraries in parallel. One could have a constant Trp, another, a constant Glu, etc.

If two residues were held constant and both were required for binding, then the incidence of binders would be increased by a much larger amount. The incidence of occurrence is independent at each position , therefore holding two residues constant is multiplicative: in a simple case of equal representation, 20 fold for each site or 400 fold overall. Evidence supporting this was found in the use of a two residue biased library to enrich for peptides which bind to src homology 3 domains (SH3) (Proc. Natl. Acad. Sci. USA. 93:1540-1544 (1996) Distinct ligand preferences of Src homology 3 domains from Src, Yes, Abl, Cortactin, p53 bp2, PLCgamma, Crk, and Grb2. A. Sparks, J. Rider, N. Hoffman, D. Fowlkes, L. Quilliam, and B. Kay). The authors found an increase in the titers of SH3-binding phage approximately 100 fold over random libraries of the same size and complexity. This is close to the theoretical increase for these libraries ((2 codons for P divided by 31 possible codons)²=240 fold increase).

In the present invention, if the library is biased at two positions, either one or both positions may be encoded by a suppressible codon. If both positions are so encoded, then either the same or different codons may be used. If the codons are the same, then the encoded AA (in a suppressor strain context) will be the same at both positions. If they are different, then these positions are independently determined by the choice of a suitable strain suppressing both codons as desired.

The use of libraries biased at two positions known to be required for binding is an extremely powerful tool. However, to make parallel biased libraries which collectively include all eleven amino acid peptides, with, in each individual biased library, two constant residues, would require passing 110 libraries (11 positions for fixed residue 1×10 positions for fixed residue 2×) through 400 (20 for position 1 times 20 for position 2) different suppressor strains, or 880 libraries through 200 different suppressor strains, etc., for a total of 44,000 possibilities. (There being a tradeoff between the number of libraries and the number of strains.) Even if one of the constant residues were always the middle residue, there would be 4,000 possibilities. While screening this number of possibilities may be possible, the increase in the number of binding peptides would probably not justify the complexities of the task.

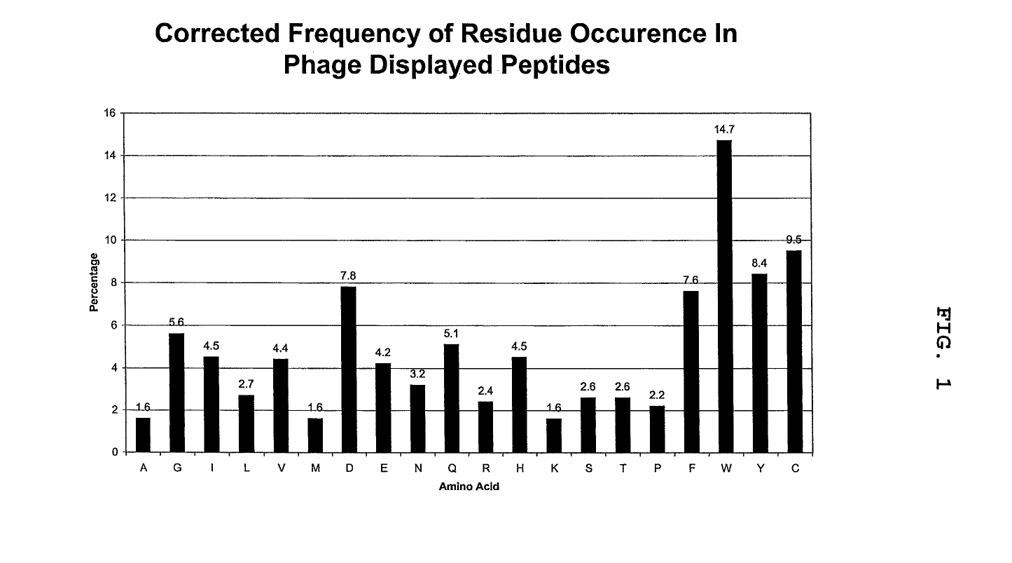

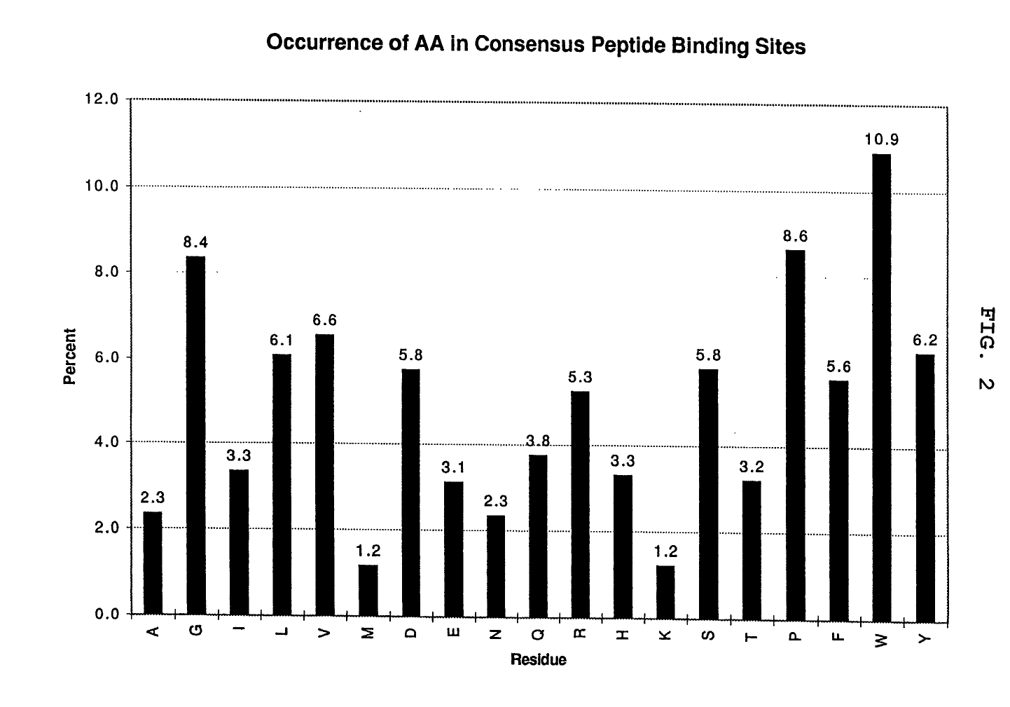

It is desirable to enrich for residues that are important for protein-peptide interactions. These residues contain side chains that can interact with other amino acids and are less likely to pack tightly, allowing a greater degree of freedom for interaction with other ligands. A study of residues at protein binding sites showed an overrepresentation of R, H, W, and Y (Villar and Kauvar, FEBS Letters 349: 125-130 (1994) Amino acid preferences at protein binding sites). A compilation of peptide sequences derived from the phage display against a series of proteins reveals that the amino acids are not found in equal amounts, that is to say that some amino acids appear in peptides that bind to various targets more frequently than other amino acids. A graph which shows the raw incidence of residue occurrence in peptides binding to any of 16 proteins is shown in FIG. 1; FIG. 2 shows the effect of correcting for codon usage. There is a clear overrepresentation of aromatic residues, proline, cysteine and aspartic acid. Biased libraries with these residues fixed or scanning through the displayed peptide are preferred, whereas biased libraries with residues that are underrepresented (such as alanine, methionine, and lysine) are less preferred, with libraries containing the remaining residues as fixed or scanning residues are of intermediate interest. As new peptides are described for additional targets, this data set should be updated and reevaluated. Nonetheless, the trends are quite clear.

An empirical way of determining which residues are preferred would be to take a representative mixture of proteins and bind to them a random synthetic peptide library. After washing away the peptides that did not bind, the remaining peptides could be eluted and the molar ratio of residues remaining bound could be determined. The profile should tell which residues result in peptides which would bind to the original mixture of proteins. This approach would also work on an individual target, providing initial information on residues important for binding. An alternative method for determining which residues are preferred would be to take the mixture of proteins and use a set of phage display libraries in which one residue of the displayed peptide is fixed to select for binding phage. After several rounds of affinity selection, the libraries with the greatest number of binding phage should be those where the fixed residue is contributing to the binding of the displayed peptides.

While certain synthetic strategies have been discussed above, the present invention is not limited, other than vis-a-vis use of a suppressible codon, to any particular method of synthesizing a combinatorial peptide library with one or more predetermined positions held constant, or with a particular mixture of amino acids at a given position.

Biological Synthesis of Peptide Libraries

A peptide library may be prepared by biological or nonbiological synthesis methods; the present invention requires use of a biological method. In a biological synthesis method, a gene encoding the peptides of interest is expressed in a host cell so that the peptides are displayed either on the surface of the cell or on the outer coat of phage produced by the cell. Of course, to achieve diversity, the gene must be randomized at those codons corresponding to variable residues of the peptide. It thus is not a single DNA, but rather a DNA mixture, which is introduced into the host cell culture, so that each cell has the potential, depending on which DNA it receives, of expressing any of the many possible peptide sequences of the library. (On average, each cell will express only one of the sequences of the mixture.) The gene may be randomized by, in the course of synthesis, using a mixture of nucleotides rather than a pure nucleotide during appropriate synthetic cycles. The synthesis cycles may add one base at a time, or an entire codon.

In screening phage libraries, it is also routine to immobilize the TP on a solid support, since nonbinding phage can be removed. (Science 249: 404-6 (1990) [90333257], Random peptide libraries: a source of specific protein binding molecules, J. J. Devlin, L. C. Panganiban & P. E. Devlin; Science 249: 386-90 (1990) [90333256], Searching for peptide ligands with an epitope library, J. K. Scott & G. P. Smith; Gene 128: 59-65 (1993)[93285470], An M13 phage library displaying random 38-amino-acid peptides as a source of novel sequences with affinity to selected targets, B. K. Kay, N. B. Adey, Y. S. He, J. P. Manfredi, A. H. Mataragnon & D. M. Fowlkes).

A structured panel of biased peptide libraries may be prepared by cloning the DNA mixture comprising the suppressible stop codon into a plurality of different suppressor strains, simultaneously or sequentially. Alternatively, phage from one library may be used to infect a different suppressor strain to obtain a new library belonging to the same structured panel. The libraries of a structured panel may be synthesized and screened in any order.

Target

The target may be any material, whether a unitary compound or a mixture or composite of some kind, for which it is desirable to find a binding peptide. Suitable molecular targets include peptides, proteins, carbohydrates, lipids and combinations thereof (e.g., glyloproteins), other organic compounds, organo-metallic compounds, and minerals. Suitable composite targets include cells, tissues and organs. Suitable mixtures include biological fluids such as blood, urine, cerebrospinal fluid and semen, and extracts of plant and animal tissues, as well as nonbiological fluids such as waste waters, and rocks or minerals.

If the target is a protein, the target protein may be a naturally occurring protein, or a subunit or domain thereof, from any natural source, including a virus, a microorganism (including bacterial, fungi, algae, and protozoa), an invertebrate (including insects and worms), or the normal or cancerous cells of a vertebrate (especially a mammal, bird or fish and, among mammals, particularly humans, apes, monkeys, cows, pigs, goats, llamas, sheep, rats, mice, rabbits, guinea pigs, cats and dogs). Alternatively, the target protein may be a mutant of a natural protein. Mutations may be introduced to facilitate the labeling or immobilization of the target protein, or to alter its biological activity (An inhibitor of a mutant protein may be useful to selectively inhibit an undesired activity of the mutant protein and leave other activities substantially intact).

The target protein may be, inter alia, a glyco-, lipo-, phosphor or metalloprotein. It may be a nuclear, cytoplasmic, membrane, or secreted protein. It may, but need not, be an enzyme. The known binding partners (if any) of the target protein may be, inter alia, other proteins, oligo- or polypeptides, nucleic acids, carbohydrates, lipids, or small organic or inorganic molecules or ions. The biological activity or function of the target protein may be, but is not limited to, being a

kinase

protein kinase

tyrosine kinase

Threonine kinase

Serine Kinase

nucleotide kinase

polynucleotide kinase

Phosphatase

Protein phosphatase

nucleotide phosphatase

acid phosphatase

alkaline phosphatase

pyrophosphatase

deaminase

protease

endoprotease

exoprotease

metalloprotease

serine endopeptidase

cysteine endopeptidase

nuclease

Deoxyribonuclease

ribonuclease

endonulcease

exonuclease

polymerase

DNA Dependent RNA polymerase

DNA Dependent DNA polymerase

telomerase

primase

Helicase

Dehydrogenase

transferase

peptidyl transferase

transaminase

glycosyltransferase

ribosyltransferase

acetyltransferase

Hydrolase

urease

carboxylase

isomerase

dismutase

rotase

topoisomerase

glycosidase

endoglycosidase

exoglycosidase

deaminase

lipase

esterase

sulfatase

cellulase

lyase

reductase

synthetase

Ion Channel

DNA Binding

RNA Binding

Ligase

RNA ligase DNA ligase

Adaptor or scaffolding protein

Structural protein

fibrin(ogen)

collagen

elastin

talin

Tumor Suppressor

adhesion molecule

oxygenase

oxidase

peroxidase

chaperonin

Transporter

electron transporter

protein transporter

peptide transporter

hormone transporter

serotonin

DOPA

nucleic acid transporter

signal transduction

neurotransmitter

structural component

of viruses

of cells

of organs

of organisms

information carrier/storage

antigen recognition protein

MHC I complex

MHC II complex

receptor

TNfα Receptor

TNFβ Receptor

β-Adrenergic Receptor

α-Adrenergic Receptor

IL-8 Receptor

IL-3 Receptor

CSF Receptor

Erythropoietin Receptor

FAS Ligand Receptor

T-cell Receptors

B-Cell Antigen Receptor

F episilon Receptor

Growth Hormone Receptor

Nuclear Receptors

Glucocorticoid

Estrogen

Testosterone

The binding protein may have more than one paratope and they may be the same or different. Different paratopes may interact with epitopes of different binding partners. An individual paratope may be specific to a particular binding partner, or it may interact with several different binding partners. A protein can bind a particular binding partner. through several different binding sites. The binding sites may be continuous or discontinuous (vis-a-vis the primary sequence of the protein).

### REFERENCES CITED

Bienz, M; Kubli, E; Kohli, J; de Henau, S; Grosjean, H. 1980. Nonsense suppression in eukaryotes: the use of the Xenopus oocyte as an in vivo assay system. Nucleic Acids Res. 8(22): 5169-5178.

Dingermann, T; Reindl, N; Brechner, T; Werner, H; Nerke, K. Nonsense suppression in Dictyostelium discoideum. Dev Genet 1990; 11(5-6): 410-417.

Franklin, S; Lin T Y; Folk, W R. 1992. Construction and expression of nonsense suppressor tRNAs which function in plant cells. Plant J 2(4):583-588.

Kondo, K; Hodgkin, J; Waterson R H. 1988. Differential expression of five tRNA(UAGTrp) amber suppressors in Caenorhabditis elegans. Mol Cell Biol 8(9):3627-3635.

Laski, F A; Belagaje, R; Hudziak, R M; Capecchi, M R; Norton, G P; RajBhandary, U L; Sharp, Pa. 1984. Synthesis of an ochre suppressor tRNA gene and expression in mammalian cells. EMBO J 3(11):2445-2452.

Laski, F A, Ganguly, S; Sharp, Pa., RajBhandary, U L; Rubin, G M. 1989 Construction, stable transformation, and function of an amber suppressor tRNA in Drosophilia melanogaster. Proc Natl Acad Sci USA 86(17)6696-6698.

Liebman, S W; Sherman, F; Stewart, J W. 1976. Isolation and characterization of amber suppressors in yeast. Genetics 82(2): 251-272.

Hoogenboom et al., Nucleic Acid Res. 19:4133-4137 (1991) Lowman et al., Biochemistry 30:10832-10838 (1991)

Felici et al., J. Mol. Biol. 222:301-310 (1991)

Miller, J. H., L. G. Kleina, J-M. Masson, J. Normanly and J. Abelson, 1989, Genome 31:905-908.

Kleina, L. G., J-M. Masson, J. Normanly, J. Abelson, and J. H. Miller, 1990, J. Mol. Biol. 213:705-717.

Normanly, J., L. G. Kleina, J-M. Masson, J. Abelson, and J. H. Miller, 1990, J. Mol. Biol. 213:719-726.

Miller, J. H., 1991, Methods Enzymol. 108:543-563.

1. A mixture of DNA molecules, each DNA molecule comprising a nucleotide sequence encoding a peptide member of a combinatorial biased peptide library, each peptide member comprising at least three amino acids, said mixture collectively encoding all peptide members of said library, said nucleotide sequence comprising a suppressible stop codon, located such that said stop codon, when suppressed, encodes an amino acid of said peptide member, said amino acid being constant and in the same position relative to the amino terminal for all peptides in said library, where at least one such suppressible stop codon is located so as to encode an amino acid of each of said peptide members other than a carboxy terminal amino acid of each of said peptide members.

2. A culture for the expression of a combinatorial biased peptide library, said culture comprising a plurality of transformed cells, each cell either displaying a peptide member of said library on its cell surface, or producing virus which display a peptide member of said library on the viral coat, said cells of said culture collectively providing for the display of the entire library, said cells having been transformed with the mixture of claim 1, and said cells suppressing said stop codon, so that said library is displayed.

3. A kit for screening peptides for target binding activity which comprises (a) a DNA mixture according to claim 1, (b) a first cell culture comprising cells which suppress said stop codon to encode a first amino acid, and (c) a second cell culture comprising cells which suppress said stop codon to encode a second amino acid, the first and second amino acids being different.

4. A method of screening peptides for binding to a target which comprises (a) providing a DNA mixture according to claim 1, (b) transforming a first cell culture with said mixture to obtain cells which suppress said stop codon to encode a first amino acid, thereby obtaining a first library, (c) transforming a second cell culture with the same mixture to obtain cells which suppress said stop codon to encode a second and different amino acid, thereby obtaining a second and different library, the first and second libraries together forming a structured panel of combinatorial libraries, and (d) screening the first and second libraries for peptides with target binding activity.

5. A mixture of virus, each virus displaying a peptide member of a combinatorial biased peptide library, said display occurring as a result of expression, in each cell infected by said virus, of a nucleotide sequence encoding said peptide member, said peptide member comprising at least three amino acids, said nucleotide sequence comprising a suppressible stop codon located such that said stop codon, when suppressed, encodes an amino acid of said peptide member, said amino acid being constant and in the same position relative to the amino terminal for all peptides in said library, said cell having so suppressed said stop codon; said mixture of virus collectively displaying the entire library,
said mixture of virus having been obtained by cultivation of the culture of claim 1 in such manner that such virus are produced,
where at least one such suppressible stop codon is located so as to encode an amino acid of each of said peptide members other than a carboxy terminal amino acid of each of said peptide members.

6. The mixture of claim 1 in which said peptides have the formula
(Xaa)ₘ-AA₁-(Xaa)ₙ
where Xaa is (a) any genetically encoded amino acid, or (b) any genetically encoded amino acid except cysteine, the Xaa may be the same or different for each amino acid position, m and n are chosen independently from the range of 2 to 20, and
AA₁ is a genetically encoded amino acid encoded by said suppressible stop codon.

7. The mixture of claim 1, said mixture being obtained by stepwise addition of nucleotides to form each DNA molecule.

8. The mixture of claim 1 wherein the suppressible stop codon is an amber (TAG) codon.

9. The method of claim 4 wherein the suppressible stop codon is an amber (TAG) codon.

10. The method of claim 9 wherein the first and second amino acids encoded by said suppressible stop codons in said first and second cultures are selected independently from the group consisting of serine, glutamine, tyrosine, lycine, leucine, glycine, alanine, cysteine, glutamic acid, histidine, phenylalanine, arginine and tryptophan.

11. The mixture of claim 1 where said nucleotide sequence comprises a suppressible amber stop codon and a suppressible opal stop codon.

12. The method of claim 4 where said nucleotide sequence comprises a suppressible amber stop codon and a suppressible opal stop codon.

13. The method of claim 4 in which only one stop codon is suppressed.

14. The mixture of claim 1 in which the peptide library has a diversity of at least 10³.

15. The method of claim 4 in which the peptide library has a diversity of at least 10³.

16. The mixture of claim 1 in which the peptide library has a diversity of between 10³ and 10⁹.

17. The method of claim 4 in which the peptide library has a diversity of between 10³ and 10⁹.

18. The mixture of claim 1 in which the peptide members have a length of 5-41 amino acids.

19. The method of claim 4 in which the peptide members have a length of 5-41 amino acids.

20. The mixture of claim 1 in which the peptide members have a length of 9 to 19 amino acids.

21. The method of claim 4 in which the peptide members have a length of 9 to 19 amino acids.

22. A method of screening peptides for binding to a target which comprises:
(a) providing a mixture of virus according to claim 5, each cell being of a first cell culture and the amino acid encoded by said suppressible stop codon as a result of expression in the first cell culture being a first amino acid;
(b) transforming a second and different cell culture with the mixture of virus of (a) above, the amino acid encoded by said suppressible step codon as a result of expression in said cell culture being a second and different amino acid; and
(c) screening the mixture of virus produced by said second cell culture for virus which display peptides which bind the target.

23. The method of claim 22 in which the mixture of virus of (a) above is also screened for display of a peptide which binds said target.

24. The method of claim 23 in which only those viruses of (a) above which display a peptide which binds said target are used to transform the second cell culture of (b) above.

25. The mixture of claim 22 in which said peptides have the formula
(Xaa)ₘ-AA₁-(Xaa)ₙ
where Xaa is (a) any genetically encoded amino acid, or (b) any genetically encoded amino acid except cysteine, the Xaa may be the same or different for each amino acid position, m and n are chosen independently from the range of 2 to 20, and
AA₁ is a genetically encoded amino acid encoded by said suppressible stop codon.

26. The method of claim 22 wherein the suppressible stop codon is an amber (TAG) codon.

27. The method of claim 26 wherein the first and second amino acids encoded by said suppressible stop codons in said first and second cultures are selected independently from the group consisting of serine, glutamine, tyrosine, lycine, leucine, glycine, alanine, cysteine, glutamic acid, histidine, phenylalanine, arginine and tryptophan.

28. The method of claim 22 where said nucleotide sequence comprises a suppressible amber stop codon and a suppressible opal stop codon.

29. The method of claim 22 in which only one stop codon is suppressed.

30. The method of claim 22 in which the peptide library has a diversity of at least 10³.

31. The method of claim 22 in which the peptide library has a diversity of between 10³ and 10⁹.

32. The method of claim 22 in which the peptide members have a length of 5-41 amino acids.

33. The method of claim 22 in which the peptide members have a length of 9 to 19 amino acids.

34. The method of claim 4 in which the libraries are libraries of viruses produced by said cell cultures, which viruses display said peptides on their coats.

35. The method of claim 4 in which the libraries are libraries of cells which display said peptides on their membranes.

36. The culture of claim 3 in which the libraries are libraries of viruses produced by said cell cultures, which viruses display said peptides on their coats.

37. The culture of claim 3 in which the libraries are libraries of cells which display said peptides on their membranes.

38. The mixture of claim 5 in which the virus are phage.

39. The mixture of claim 5 in which the virus are filamentous phage.

40. The method of claim 34 in which the virus are phage.

41. The method of claim 34 in which the virus are filamentous phage.

42. The method of claim 22 in which the virus are phage.

43. The method of claim 22 in which the virus are filamentous phage.

44. The method of claim 4 in which the cell culture is a bacterial cell culture.

45. The method of claim 22 in which the cell culture is a bacterial cell culture.

46. The method of claim 4 in which the culture is of Escherichia coli cells.

47. The method of claim 22 in which the culture is of Escherichia coli cells.

48. The culture of claim 3 in which the cells are yeast cells, and the yeast cells express both a gene encoding said peptide members, when said stop codon is suppressed, and a suitable suppressor gene.

49. The method of claim 4 in which the cell cultures are of yeast cells.

50. The culture of claim 2 in which the libraries are libraries of viruses produced by said cell cultures, which viruses display said peptides on their coats.

51. The culture of claim 2 in which the libraries are libraries of cells which display said peptides on their membranes.

52. The culture of claim 2 in which the cells are yeast cells, and the yeast cells express both a gene encoding said peptide members, when said stop codon is suppressed, and a suitable suppressor gene.
